SEQUENCE LISTING (11 sequences)

SEQ ID NO: 1 - protein, 5 aa, Artificial Sequence
  source: /note=“Description of Artificial Sequence Synthetic peptide”
SYYMN

SEQ ID NO: 2 - protein, 17 aa, Artificial Sequence
  source: /note=“Description of Artificial Sequence Synthetic peptide”
GISGDPSNTYYADSVKG

SEQ ID NO: 3 - protein, 11 aa, Artificial Sequence
  source: /note=“Description of Artificial Sequence Synthetic peptide”
DLPLVYTGFAY

SEQ ID NO: 4 - protein, 11 aa, Artificial Sequence
  source: /note=“Description of Artificial Sequence Synthetic peptide”
SGDNLRHYYVY

SEQ ID NO: 5 - protein, 7 aa, Artificial Sequence
  source: /note=“Description of Artificial Sequence Synthetic peptide”
GDSKRPS

SEQ ID NO: 6 - protein, 9 aa, Artificial Sequence
  source: /note=“Description of Artificial Sequence Synthetic peptide”
QTYTGGASL

SEQ ID NO: 7 - protein, 120 aa, Artificial Sequence
  source: /note=“Description of Artificial Sequence Synthetic polypeptide”
QVQLVESGGGLVQPGGSLRLSCAASGFTFSSYYMNWVRQAPGKGLEWVSGISGDPSNTYYADSVKGRFTISRDNSKNTLYLQMNSLRAEDTAVYYCARDLPLVYTGFAYWGQGTLVTVSS

SEQ ID NO: 8 - protein, 109 aa, Artificial Sequence
  source: /note=“Description of Artificial Sequence Synthetic polypeptide”
DIELTQPPSVSVAPGQTARISCSGDNLRHYYVYWYQQKPGQAPVLVIYGDSKRPSGIPERFSGSNSGNTATLTISGTQAEDEADYYCQTYTGGASLVFGGGTKLTVLGQ

SEQ ID NO: 9 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 10 - DNA, 360 bp, Artificial Sequence
  source: /note=“Description of Artificial Sequence Synthetic polynucleotide”
caggtgcaattggtggaaagcggcggcggcctggtgcaaccgggcggcagcctgcgtctgagctgcgcggcctccggatttaccttttcttcttattatatgaattgggtgcgccaagcccctgggaagggtctcgagtgggtgagcggtatctctggtgatcctagcaatacctattatgcggatagcgtgaaaggccgttttaccatttcacgtgataattcgaaaaacaccctgtatctgcaaatgaacagcctgcgtgcggaagatacggccgtgtattattgcgcgcgtgatcttcctcttgtttatactggttttgcttattggggccaaggcaccctggtgacggttagctca

SEQ ID NO: 11 - DNA, 327 bp, Artificial Sequence
  source: /note=“Description of Artificial Sequence Synthetic polynucleotide”
gatatcgaactgacccagccgccttcagtgagcgttgcaccaggtcagaccgcgcgtatctcgtgtagcggcgataatcttcgtcattattatgtttattggtaccagcagaaacccgggcaggcgccagttcttgtgatttatggtgattctaagcgtccctcaggcatcccggaacgctttagcggatccaacagcggcaacaccgcgaccctgaccattagcggcactcaggcggaagacgaagcggattattattgccagacttatactggtggtgcttctcttgtgtttggcggcggcacgaagttaaccgttcttggccag

(19) United States
(12) Patent Application Publication
(10) Pub. No.: US 2024/0132618 A1
(43) Pub. Date: Apr. 25, 2024

(54) ANTI-CD38 ANTIBODIES FOR USE IN THE TREATMENT OF ANTIBODY-MEDIATED TRANSPLANT REJECTION

(71) Applicant: MORPHOSYS AG, Planegg (DE)

(72) Inventors: Stefan Steidl, Munich (DE); Stefan Härtle, Mammendorf (DE); Rainer Boxhammer, Kolbermoor (DE)

(21) Appl. No.: 18/548,477

(22) PCT Filed: Mar. 1, 2022
(86) PCT No.: PCT/EP2022/055080

§ 371 Date: Aug. 29, 2023

(30) Foreign Application Priority Data
Mar. 1, 2021 (EP) 21159860.2

(51) Int. Cl. C07K 16/28

(52) U.S. Cl. CPC C07K 16/2896; A61K 2039/545; C07K 2317/24; C07K 2317/52; C07K 2317/565

(57) ABSTRACT

The present invention relates to the use of the anti-CD38 antibody felzartamab in the prophylaxis and/or treatment of antibody-mediated rejection (ABMR) of transplants. In accordance with the present invention, felzartamab is effective in the treatment of antibody-mediated renal allograft rejection.

### FIELD OF THE INVENTION

The present disclosure relates to the field of organ transplantation (e.g. kidney transplantation). In particular, the present disclosure relates to anti-CD38 antibodies for use in the treatment of patients with antibody-mediated transplant rejection (ABMR). The disclosure provides methods for the reduction of antibody-secreting cells and for the decrease of antibody levels with specificities directed against one or more antigen(s) present on the transplanted organ, using an anti-CD38 antibody. In accordance with the present invention, an anti-CD38 antibody, alone or in combination with one or more immunosuppressive drugs, can be effective in the treatment and/or prophylaxis of ABMR. An anti-CD38 antibody for use according to the present invention includes felzartamab (MOR202).

### BACKGROUND

Organ transplantation is a medical procedure in which an organ is removed from the body of one subject (donor) and placed in the body of a recipient (host), to replace a damaged or missing organ. Transplantation is the treatment of choice for patients developing end stage organ failure. Primarily transplants between two subjects of the same species are performed (so-called allografts) to reduce organ rejection by the immune system of the host. However, the host immune system recognizes even well matched transplants and eventually may destroy the transplant. Formerly, it was held that alloreactive T cells to be solely responsible for graft injury by T cell mediated rejection (TCMR). In the meantime, it is established that anti-donor alloantibodies are an additional important barrier to long-term graft survival. This so-called antibody-mediated rejection (ABMR) often contributes to graft loss after organ transplantation. Anti-donor-specific antibodies (DSA), e.g. anti-human leukocyte antigen (HLA) antibodies, are a major trigger for chronic graft injury possibly in combination with antibody-mediated activation of cellular mechanisms (e.g., activation of natural killer cells). In kidney transplantation, ABMR is one of the main causes of allograft dysfunction and chronic allograft injury. The rejection of the transplanted kidney, commonly triggered by anti-HLA DSA, is associated with a progressive decline in glomerular filtration rate (GFR), increased proteinuria, and kidney failure.

Many studies exist in the prior art evaluating different treatment strategies for ABMR. Known strategies comprise, for example,
immunosuppression with tacrolimus, mycophenolate mofetil and belatacept (CTIA-4 Fc-fusion)(Theruvath, TP et al. 2001, Transplantation, 72:77-83; Schwarz, C et al. 2015, Transplant International, 28:820-827),
immunomodulatory measures, including high dose intravenous immunoglobulin with or without anti-CD20 rituximab administration (Fehr, Tet al. 2009, Transplantation, 87:1837- 1841),
the proteasome inhibitor bortezomib (Walsh, RC et al. 2012, Kidney Int, 81:1067-1074) or
complement inhibitors (Eskandary, F et al. 2017, Am J Transplant, 18:916-926).

However, significant improvements could not be sufficiently be achieved in the long-term course by these strategies. Therefore, treatment options for long-term graft survival still need to be improved.

One promising target may be CD38, primarily expressed on immune and hematopoietic cells, with particularly high expression levels on antibody-producing plasma cells. Considering the critical role of alloantibody-producing plasma cells in ABMR (when DSA are the cause of injury), effective plasma cell depletion via CD38 may be useful in transplantation medicine to achieve sustained DSA reduction.

The concept of counteracting ABMR with an anti-CD38 antibody has been shown in the prior art with daratumumab. In a rhesus model with kidney transplants, daratumumab reduced donor-specific antibodies and led to a prolonged renal allograft survival (Kwun, J et al. 2019, Journal of the American Society of Nephrology, 30: 1206-1219). W02020185672 (Cedars-Sinai) exemplifies two cases of patients with anti-HLA antibodies and standard-of-care resistant ABMR who received treatment with daratumumab, leading to an initial reduction of anti-HLA antibody levels.

However, a drawback of this treatment was an increase of CD4 and CD8 T cells and the elimination of regulatory B cells (B-regs) post-daratumumab treatment. This may be due to the subsidiary effect of daratumumab on the reduction of regulatory T and B cells. Thus, targeting CD38 with daratumumab not only leads to a reduction of plasma cell populations but also depletes beneficial regulatory cell populations. The presence of regulatory T cells (Tregs) within the peripheral circulation and graft microenvironment may be important in inducing and maintaining long-term graft tolerance.

Further, there was no meaningful impact on the levels of anti-HLA class II antibodies, including the DSA DQ5, with a rebound in several class II antibodies and appearance of de novo HLA class II antibodies. This may be due to daratumumab's ability to deplete CD38+natural killer (NK) cells, thus restricting ADCC. FIG. 1 shows the impact of daratumumab compared to MOR202 and isatuximab on depleting NK cells in vitro.

In summary, those studies effectively control early ABMR episodes but they show that the treatment options applied in early ABMR have limited effect on late/chronic episodes, which remain the leading cause of late graft loss.

Thus, there is a high need for novel strategies for targeting alloanti body reactivity to treat ABMR and for prolonging long-term graft survival.

The main mode of action for MOR202-induced lysis of plasma cells is ADCC and ADCP, but not CDC. CDC is believed to be a major contributor to infusion-related reactions. Therefore, a major advantage, compared to other CD38 antibodies, is a lower risk of infusion-related reactions. Furthermore, MOR202 depletes mainly high CD38 cells and thereby sparing specific cell population with low CD38 levels in vitro. Certain regulatory cell subsets may be preserved after treatment with MOR202 resulting in an improved graft survival.

The present disclosure provides the anti-CD38 antibody felzartamab for use in an efficient, safe, sustainable and well-tolerated strategy in managing ABMR, in particular late and/or chronic ABMR. Repeated administration of felzartamab is able to counteract tissue inflammation (i.e. the increase of CD4+and CD8+T cell numbers) and graft injury in ongoing ABMR, in particular, inflammation in the microcirculation, B cell responses to HLA antigen and, as a consequence, alloantibody/NK cell-triggered chronic graft injury.

### SUMMARY OF THE INVENTION

The present invention provides the anti-CD38 antibody felzartamab for use in the treatment and/or prevention of antibody-mediated rejection of an organ transplant. Furthermore, methods of reducing or removing donor specific antibodies (e.g. anti-HLA), and/or treating or reducing the severity of ABMR in a subject who received a kidney transplant are provided. The methods include administering to the patient an effective amount of the anti-CD38 antibody felzartamab. In some aspects, the methods further include selecting a patient experiencing or having experienced ABMR of an organ transplant. In other aspects, the methods further include selecting a patient with anti-HLA antibodies in the serum that are specific to the donors HLA.

### BRIEF DESCRIPTION OF THE DRAWINGS

FIG. 1: Specific killing in vitro of a CD38 high expressing MM plasma cell line by MOR202 while sparing CD38 low expressing NK cells compared to daratumumab (Dara) and isatuximab.

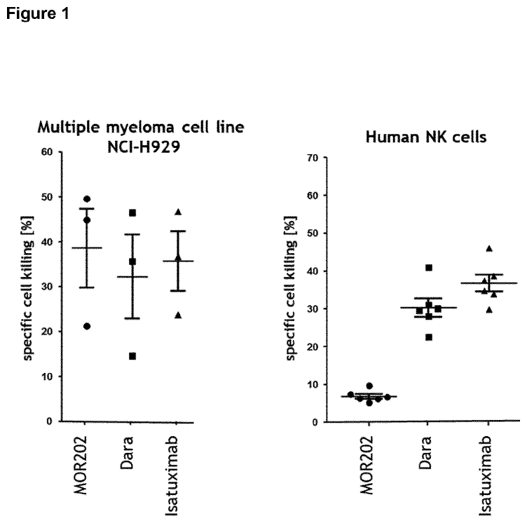

FIG. 2: Scheme of the phase 2 pilot trial of felzartamab in late ABMR.

### DETAILED DESCRIPTION OF THE INVENTION

### Definitions

The term “CD38” refers to a protein known as CD38, having the following synonyms: ADP-ribosyl cyclase 1, cADPr hydrolase 1, Cyclic ADP-ribose hydrolase 1, T10. Human CD38 (UniProt P28907) has the following amino acid sequence:

(SEQ ID NO.: 9)
MANCEFSPVSGDKPCCRLSRRAQLCLGVSILVLILVVVLAVVVPRWRQQ
WSGPGTTKRFPETVLARCVKYTEIHPEMRHVDCQSVWDAFKGAFISKHP
CNITEEDYQPLMKLGTQTVPCNKILLWSRIKDLAHQFTQVQRDMFTLED
TLLGYLADDLTWCGEFNTSKINYQSCPDWRKDCSNNPVSVFWKTVSRRF
AEAACDVVHVMLNGSRSKIFDKNSTFGSVEVHNLQPEKVQTLEAWVIHG
GREDSRDLCQDPTIKELESIISKRNIQFSCKNIYRPDKFLQCVKNPEDS
SCTSEI

CD38 is a type II transmembrane glycoprotein and an example of an antigen that is highly expressed on antibody-secreting cells (e.g.: plasmablasts and plasma cells). Functions ascribed to CD38 include both receptor-mediated adhesion and signaling events and (ecto-) enzymatic activity. As an ectoenzyme, CD38 uses NAD+ as substrate for the formation of cyclic ADP-ribose (cADPR) and ADPR, but also of nicotinamide and nicotinic acid-adenine dinucleotide phosphate (NAADP). cADPR and NAADP have been shown to act as second messengers for Ca2+ mobilization. By converting NAD+to cADPR, CD38 regulates the extracellular NAD+ concentration and hence cell survival by modulation of NAD-induced cell death (NCI D). In addition to signaling via Ca2+, CD38 signaling occurs via cross-talk with antigen-receptor complexes on T and B cells or other types of receptor complexes, e.g. MHC molecules, and is in this way involved in several cellular responses, but also in switching and secretion of IgG antibodies.

The term “anti-0038 antibody”, as used herein, includes anti-CD38 binding molecules in its broadest sense; any molecule which specifically binds to CD38 or inhibits the activity or function of CD38, or which by any other way exerts a therapeutic effect on CD38 is included. Any molecule that interferes or inhibits CD38 functionality is included. The term “anti-CD38 antibody” includes, but is not limited to, antibodies specifically binding to CD38, alternative protein scaffolds binding to CD38, nucleic acids (including aptamers) specific for CD38 or small organic molecules specific for CD38.

Antibodies specific for CD38 are described for example in W0199962526 (Mayo Foundation); W0200206347 (Crucell Holland); US2002164788 (Jonathan Ellis); W02005103083, W02006125640, W02007042309 (MorphoSys), W02006099875 (Genmab), and W02008047242 (Sanofi-Aventis). Combinations of antibodies specific for CD38 and other agents are described for example in W0200040265 (Research Development Foundation); W02006099875 and W02008037257 (Genmab); and W02010061360, W02010061359, W02010061358 and W02010061357 (Sanofi Aventis). CD38-targeting antibodies are broadly used in multiple myeloma (reviewed in Frerichs KA et al. 2018, Expert Rev Clin Immuno1;14(3):197-206). Further uses of anti-CD38 antibodies are described for example in W02015130732, W02016089960, W02016210223 (Janssen), W02018002181 (UMC Utrecht), W02019020643 (ENCEFA), W02020185672 (Cedars-Sinai) and W02020187718 (MorphoSys) which are all incorporated by reference in their entireties.

Preferably, an anti-CD38 antibody for the use as described herein is an antibody specific for CD38. More preferably, an anti-CD38 antibody is an antibody or antibody fragment, such as a monoclonal antibody, specifically binding to CD38 and deleting specific CD38 positive B cells, plasma cells, plasmablasts and any other CD38 positive antibody-secreting cells. Such an antibody may be of any type, such as a murine, a rat, a chimeric, a humanized or a human antibody.

A “human antibody” or “human antibody fragment”, as used herein, is an antibody or antibody fragment having variable regions in which the framework and CDR regions are from sequences of human origin. If the antibody contains a constant region, the constant region also is from such sequences. Human origin includes, but is not limited to human germline sequences, or mutated versions of human germline sequences or antibody containing consensus framework sequences derived from human framework sequences analysis, for example, as described in Knappik et al., (2000) J Mol Biol 296:57-86). Human antibodies can be isolated e.g. from synthetic libraries or from transgenic mice (e.g. Xenomouse). An antibody or antibody fragment is human if its sequence is human, irrespective of the species from which the antibody is physically derived, isolated, or manufactured.

The structures and locations of immunoglobulin variable domains, e.g., CDRs, may be defined using well known numbering schemes, e.g., the Kabat numbering scheme, the Chothia numbering scheme, or a combination of Kabat and Chothia (see, e.g. Sequences of Proteins of Immunological Interest, U.S. Department of Health and Human Services (1991), eds. Kabat et al.; Lazikani et al., (1997) J. Mol. Bio. 273:927-948); Kabat et al., (1991) Sequences of Proteins of Immunological Interest, 5th edit., NIH Publication no. 91-3242 U.S. Department of Health and Human Services; Chothia et al., (1987) J. Mol. Biol. 196:901-917; Chothia et al., (1989) Nature 342:877-883; and Al-Lazikani et al., (1997) J. Mol. Biol. 273:927-948.

A “humanized antibody” or “humanized antibody fragment” is defined herein as an antibody molecule, which has constant antibody regions derived from sequences of human origin and the variable antibody regions or parts thereof or only the CDRs are derived from another species. For example, a humanized antibody can be CDR-grafted, wherein the CDRs of the variable domain are from a non-human origin, while one or more frameworks of the variable domain are of human origin and the constant domain (if any) is of human origin.

The term “chimeric antibody” or “chimeric antibody fragment” is defined herein as an antibody molecule, which has constant antibody regions derived from, or corresponding to, sequences found in one species and variable antibody regions derived from another species. Preferably, the constant antibody regions are derived from, or corresponding to, sequences found in humans, and the variable antibody regions (e.g. VH, VL, CDR or FR regions) are derived from sequences found in a non-human animal, e.g. a mouse, rat, rabbit or hamster.

The term “isolated antibody” refers to an antibody or antibody fragment that is substantially free of other antibodies or antibody fragments having different antigenic specificities. Moreover, an isolated antibody or antibody fragment may be substantially free of other cellular material and/or chemicals. Thus, in some aspects, antibodies provided are isolated antibodies, which have been separated from antibodies with a different specificity. An isolated antibody may be a monoclonal antibody. An isolated antibody may be a recombinant monoclonal antibody. An isolated antibody that specifically binds to an epitope, isoform or variant of a target may, however, have cross-reactivity to other related antigens, e.g., from other species (e.g., species homologs).

The term “monoclonal antibody” as used herein refers to a preparation of antibody molecules of single molecular composition. A monoclonal antibody composition displays a unique binding site having a unique binding specificity and affinity for particular epitopes.

In addition, as used herein, an “immunoglobulin” (Ig) hereby is defined as a protein belonging to the class IgG, IgM, IgE, IgA, or IgD (or any subclass thereof), and includes all conventionally known antibodies and functional fragments thereof.

The phrase “antibody fragment”, as used herein, refers to one or more portions of an antibody that retain the ability to specifically interact with (e.g., by binding, steric hindrance, stabilizing spatial distribution) an antigen. Examples of binding fragments include, but are not limited to, a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment, which consists of a VH domain; and an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as “single chain Fragment (scFv)”). Such single chain antibodies are to be encompassed within the term “antibody fragment”. Antibody fragments can also be incorporated into single domain antibodies, maxibodies, minibodies, intrabodies, diabodies, triabodies, tetrabodies, v-NAR and bis-scFv. Antibody fragments can be grafted into scaffolds based on polypeptides such as Fibronectin type III (Fn3). Antibody fragments can be incorporated into single chain molecules comprising a pair of tandem Fv segments (VH-CH1-VH-CH1) which, together with complementary light chain polypeptides, form a pair of antigen-binding sites).

The present disclosure provides therapeutic methods comprising the administration of a therapeutically effective amount of an anti-CD38 antibody as disclosed to a subject in need of such treatment. A “therapeutically effective amount” or “effective amount”, as used herein, refers to the amount of an antibody specific for CD38, necessary to elicit the desired biological response. In accordance with the present disclosure, the therapeutic effective amount is the amount of an antibody specific for CD38 necessary to treat and/or prevent immune complex mediated diseases and symptoms associated with said diseases. An effective amount for a particular individual may vary, depending on factors such as the condition being treated, the overall health of the patient, the method route and dose of administration and the severity of side effects (Maynard, et al. (1996) A Handbook of SOPs for Good Clinical Practice, Interpharm Press, Boca Raton, Fla.; Dent (2001) Good Laboratory and Good Clinical Practice, London, UK).

As used herein, the terms “treat”, “treating”, treatment” or the like, mean to alleviate symptoms, eliminate the causation of symptoms either on a temporary or permanent basis, or to prevent or slow the appearance of symptoms of the named disorder or condition. They refer to both therapeutic treatment and prophylactic or preventative measures. Objectives of a treatment are to prevent or slow down (lessen) an undesired physiological change or disorder or to cure the disease to be treated. Beneficial or desired clinical results include alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if a subject was not receiving treatment. Those in need of treatment include those already with the condition or disorder as well as those prone to have the condition or disorder or those in which the condition or disorder is to be prevented.

“Preventing” or “prevention” refers to a reduction in risk of acquiring or developing a disease or disorder (i.e. causing at least one of the clinical symptoms of the disease not to develop in a subject that may be exposed to a disease-causing agent, or predisposed to the disease in advance of disease onset. “Prevention” refers to methods which aim to prevent the onset of a disease or its symptoms or which delay the onset of a disease or its symptoms.

“Administered” or “administration” includes but is not limited to delivery of a drug by an injectable form, such as, for example, an intravenous, intramuscular, intradermal or subcutaneous route or mucosal route, for example, as a nasal spray or aerosol for inhalation or as an ingestible solution, capsule or tablet. Preferably, the administration is by an injectable form.

By co-administration is included any means of delivering two or more therapeutic agents to the patient as part of the same treatment regimen, as will be apparent to the skilled person. Whilst the two or more agents may be administered simultaneously in a single formulation, i.e. as a single pharmaceutical composition, this is not essential. The agents may be administered in different formulations and at different times. The therapies (e.g., prophylactic or therapeutic agents) of the combination therapies can be administered concomitantly (concurrently) or sequentially to a subject. The therapy (e.g., prophylactic or therapeutic agents) of the combination therapies can also be cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time and repeating this sequential administration, i.e., the cycle. This is to reduce the development of resistance to one of the therapies to avoid or reduce the side effects of one of the therapies, and/or to improve, the efficacy of the therapies. The terms “concomitantly” or “concurrently” are not limited to the administration of therapies at exactly the same time, but rather it is meant that a pharmaceutical composition comprising antibodies or antibody fragments of the disclosure are administered to a subject in a sequence and within a time interval such that the antibodies of the disclosure can act together with the other therapy(ies) to provide an increased benefit than if they were administered otherwise.

“Subject” or “species”, as used herein refers to any mammal, including rodents, such as mouse or rat, and primates, such as cynomolgus monkey (Macaca fascicularis), rhesus monkey (Macaca mulatta) or humans (Homo sapiens). Preferably, the subject is a primate, most preferably a human.

As used herein, the term “a subject in need thereof” or the like, mean a human or a non-human animal patient that exhibits one or more symptoms or indicia of antibody-mediated rejection of an organ transplantation). Preferably, the subject is a primate, most preferably a human patient who has been diagnosed with antibody-mediated rejection after kidney transplantation.

The term “antibody-mediated rejection” (“ABMR”) refers to a well-established entity, frequently occurring after organ transplantation (Tx), comprising defined diagnostic criteria according to the Banff classification, e.g. inflammation and morphological damage in the microcirculation, (non-obligatory) deposition of the complement cleavage product C4d along the graft endothelium, and detection of antibodies against donor antigens (“donor-specific antibodies”, DSA). DSA can be (i) antibodies against HLA of the donor and/or (ii) non-HLA antibodies, which may be classified into at least two main categories: alloantibodies directed against polymorphic antigens that differ between the recipient and donor and antibodies that recognize self-antigens or autoantibodies.

As used herein, the term “about” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

“Pharmaceutically acceptable” means approved or approvable by a regulatory agency of the Federal or a state government or the corresponding agency in countries other than the United States, or that is listed in the US Pharmacopoeia or other generally recognized pharmacopoeia for use in animals, and more particularly, in humans.

“Pharmaceutically acceptable vehicle” refers to a diluent, adjuvant, excipient or carrier with which an antibody or antibody fragment is administered.

Throughout this specification, unless the context requires otherwise, the words “comprise”, “have” and “include” and their respective variations such as “comprises”, “comprising”, “has”, “having”, “includes” and “including” will be understood to imply the inclusion of a stated element or integer or group of elements or integers but not the exclusion of any other element or integer or group of elements or integers.

“Felzartamab” is an anti-CD38 antibody, also known as “MOR202”, “MOR03087” or “MOR3087”. The terms are used interchangeable in the present disclosure. MOR202 has an IgG1 Fc region.

The amino acid sequence of the MOR202 HCDR1 according to Kabat is:

(SEQ ID NO: 1)
SYYMN

The amino acid sequence of the MOR202 HCDR2 according to Kabat is:

(SEQ ID NO: 2)
GISGDPSNTYYADSVKG

The amino acid sequence of the MOR202 HCDR3 according to Kabat is:

(SEQ ID NO: 3)
DLPLVYTGFAY

The amino acid sequence of the MOR202 LCDR1 according to Kabat is:

(SEQ ID NO: 4)
SGDNLRHYYVY

The amino acid sequence of the MOR202 LCDR2 according to Kabat is:

(SEQ ID NO: 5)
GDSKRPS

The amino acid sequence of the MOR202 LCDR3 is

(SEQ ID NO: 6)
QTYTGGASL

The amino acid sequence of the MOR202 Variable Heavy Domain is:

(SEQ ID NO: 7)
QVQLVESGGGLVQPGGSLRLSCAASGFTFSSYYMNWVRQAPGKGLEWVS
GISGDPSNTYYADSVKGRFTISRDNSKNTLYLQMNSLRAEDTAVYYCAR
DLPLVYTGFAYWGQGTLVTVSS

The amino acid sequence of the MOR202 Variable Light Domain is:

(SEQ ID NO: 8)
DIELTQPPSVSVAPGQTARISCSGDNLRHYYVYWYQQKPGQAPVLVIYG
DSKRPSGIPERFSGSNSGNTATLTISGTQAEDEADYYCQTYTGGASLVF
GGGTKLTVLGQ

The DNA sequence encoding the MOR202 Variable Heavy Domain is:

(SEQ ID NO: 10)
CAGGTGCAATTGGTGGAAAGCGGCGGCGGCCTGGTGCAACCGGGCGGCA
GCCTGCGTCTGAGCTGCGCGGCCTCCGGATTTACCTTTTCTTCTTATTA
TATGAATTGGGTGCGCCAAGCCCCTGGGAAGGGTCTCGAGTGGGTGAGC
GGTATCTCTGGTGATCCTAGCAATACCTATTATGCGGATAGCGTGAAAG
GCCGTTTTACCATTTCACGTGATAATTCGAAAAACACCCTGTATCTGCA
AATGAACAGCCTGCGTGCGGAAGATACGGCCGTGTATTATTGCGCGCGT
GATCTTCCTCTTGTTTATACTGGTTTTGCTTATTGGGGCCAAGGCACCC
TGGTGACGGTTAGCTCA.

The DNA sequence encoding the MOR202 Variable Light Domain is:

(SEQ ID NO: 11)
GATATCGAACTGACCCAGCCGCCTTCAGTGAGCGTTGCACCAGGTCAGA
CCGCGCGTATCTCGTGTAGCGGCGATAATCTTCGTCATTATTATGTTTA
TTGGTACCAGCAGAAACCCGGGCAGGCGCCAGTTCTTGTGATTTATGGT
GATTCTAAGCGTCCCTCAGGCATCCCGGAACGCTTTAGCGGATCCAACA
GCGGCAACACCGCGACCCTGACCATTAGCGGCACTCAGGCGGAAGACGA
AGCGGATTATTATTGCCAGACTTATACTGGTGGTGCTTCTCTTGTGTTT
GGCGGCGGCACGAAGTTAACCGTTCTTGGCCAG.

### EMBODIMENTS

### Antibody

In certain embodiments of the present disclosure, the antibody or antibody fragment specific for CD38 for the use according to the present disclosure comprises a variable heavy chain variable region, a variable light chain region, heavy chain, light chain and/or CDRs comprising any of the amino acid sequences of the CD38 specific antibodies as set forth in W02007042309.

In an embodiment, said antibody or antibody fragment specific for CD38 for the use according to the present disclosure comprises a HCDR1 region comprising the amino acid sequence of SEQ ID NO: 1, a HCDR2 region comprising the amino acid sequence of SEQ ID NO: 2, a HCDR3 region comprising the amino acid sequence of SEQ ID NO: 3, a LCDR1 region comprising the amino acid sequence of SEQ ID NO: 4, a LCDR2 region comprising the amino acid sequence of SEQ ID NO: 5 and a LCDR3 region comprising the amino acid sequence of SEQ ID NO: 6.

In one embodiment, said antibody or antibody fragment specific for CD38 for the use according to the present disclosure, comprises the HCDR1 region of SEQ ID NO: 1, the HCDR2 region of SEQ ID NO: 2, the HCDR3 region of SEQ ID NO: 3, the LCDR1 region of SEQ ID NO: 4, the LCDR2 region of SEQ ID NO: 5 and the LCDR3 region of SEQ ID NO: 6.

In an embodiment, said antibody or antibody fragment specific for CD38 for the use according to the present disclosure comprises a variable heavy chain region of SEQ ID NO: 7 and a variable light chain region of SEQ ID NO: 8.

In another embodiment the anti-CD38 antibody or antibody fragment for the use according to the present disclosure comprises a variable heavy chain region of SEQ ID NO: 7 and a variable light chain region of SEQ ID NO: 8 or a variable heavy chain region and a variable light chain region that has at least 60%, at least 70%, at least 80%, at least 90% or at least 95% identity to the a variable heavy chain region of SEQ ID NO: 7 and to the variable light chain region of SEQ ID NO: 8.

An exemplary antibody or antibody fragment for the use according to the present disclosure comprising the variable heavy chain region comprising the amino acid sequence of SEQ ID NO: 7 and a variable light chain region comprising the amino acid sequence of SEQ ID NO: 8 is the human anti-CD38 antibody known as MOR202 (felzartamab).

In one embodiment, the present disclosure refers to a nucleic acid composition comprising a nucleic acid sequence or a plurality of nucleic acid sequences encoding said antibody or antibody fragment specific for CD38 for the use according to the present disclosure, wherein said antibody or antibody fragment comprises the HCDR1 region of SEQ ID NO: 1, the HCDR2 region of SEQ ID NO: 2, the HCDR3 region of SEQ ID NO: 3, the LCDR1 region of SEQ ID NO: 4, the LCDR2 region of SEQ ID NO: 5 and the LCDR3 region of SEQ ID NO: 6.

In another embodiment, the disclosure refers to a nucleic acid encoding an isolated monoclonal antibody or fragment thereof for the use according to the present disclosure wherein the nucleic acid comprises a VH of SEQ ID NO: 10 and a VL of SEQ ID NO: 11.

In one embodiment, the disclosed antibody or antibody fragment specific for CD38 for the use according to the present disclosure is a monoclonal antibody or antibody fragment.

In one embodiment, the disclosed antibody or antibody fragment specific for CD38 for the use according to the present disclosure is a human, humanized or chimeric antibody.

In certain embodiments, said antibody or antibody fragment specific for CD38 for the use according to the present disclosure is an isolated antibody or antibody fragment.

In another embodiment, said antibody or antibody fragment for the use according to the present disclosure is a recombinant antibody or antibody fragment.

In a further embodiment, said antibody or antibody fragment for the use according to the present disclosure is a recombinant human antibody or antibody fragment.

In a further embodiment, said recombinant human antibody or antibody fragment for the use according to the present disclosure is an isolated recombinant human antibody or antibody fragment.

In a further embodiment, said recombinant human antibody or antibody fragment or isolated recombinant human antibody or antibody fragment for the use according to the present disclosure is monoclonal.

In one embodiment, the disclosed antibody or antibody fragment for the use according to the present disclosure is of the IgG isotype. In a particular embodiment, said antibody is an IgG1.

In particular aspects of the present invention, the anti-CD38 antibody for the use according to the present disclosure is MOR202 (felzartamab).

In an embodiment, the present disclosure refers to a pharmaceutical composition comprising felzartamab (MOR202) or fragment thereof specific for CD38 and a pharmaceutically acceptable carrier or excipient for the use according to the present disclosure.

In certain embodiments, said antibody or antibody fragment specific for CD38 is an isolated monoclonal antibody or antibody fragment that specifically binds to human CD38.

### Pharmaceutical Compositions

When employed as a pharmaceutical the antibody, or antibody fragment, specific for CD38 is typically administered in a pharmaceutical composition. The compositions of the present disclosure are preferably pharmaceutical compositions comprising felzartamab (MOR202) and a pharmaceutically acceptable carrier, diluent or excipient, for the use in treating, inhibiting and/or reducing the severity of an antibody-mediated rejection (ABMR) response of an organ transplant in a subject in need thereof.

The pharmaceutically acceptable carrier should be suitable for intravenous, intramuscular, subcutaneous, parenteral, spinal or epidermal administration (e.g., by injection or infusion).

Pharmaceutically carriers enhance or stabilize the composition, or facilitate the preparation of the composition. Pharmaceutically acceptable carriers include solvents, dispersion media, coatings, antibacterial and antifungal agents, isotonic and absorption delaying agents, and the like that are physiologically compatible. In many cases, it is preferable to include isotonic agents, for example, sugars, polyalcohols such as mannitol or sorbitol, and sodium chloride in the composition.

A pharmaceutical composition of the present disclosure can be administered by a variety of routes known in the art. Selected routes of administration for antibodies or antibody fragments of the disclosure include intravenous, intramuscular, intradermal, intraperitoneal, subcutaneous, spinal or other parenteral routes of administration, for example by injection or infusion.

The antibody, or antibody fragment, specific for CD38 is preferably formulated as injectable composition. In preferred aspects, the anti-CD38 antibody of the present disclosure is administered intravenously. In other aspects, the anti-CD38 antibody of the present disclosure is administered, subcutaneously, intraarticularly or intra-spinally.

An important aspect of the present disclosure is a pharmaceutical composition that is able to mediate killing of CD38-expressing antibody-secreting cells (e.g. plasmablasts, plasma cells) by ADCC and ADCP.

### Methods of Treatment

In one embodiment, the present disclosure provides an anti-CD38 antibody or antibody fragment, or a pharmaceutical composition comprising an anti-CD38 antibody or antibody fragment, for use in treating, inhibiting and/or reducing the severity of antibody-mediated rejection (ABMR) response of an organ transplant in a subject in need thereof.

In certain embodiments, the organ transplant is one or more of kidney, heart, liver, lung, pancreas, stomach, skin and intestines.

In one embodiment, an anti-CD38 antibody or antibody fragment, or a pharmaceutical composition comprising an anti-CD38 antibody or antibody fragment, for use in treating, inhibiting and/or reducing the severity of antibody-mediated rejection (ABMR) response of a kidney transplant in a subject in need thereof is provided.

In a particular embodiment, the present disclosure provides an anti-CD38 antibody or antibody fragment comprising the HCDR1 region of SEQ ID NO: 1, the HCDR2 region of SEQ ID NO: 2, the HCDR3 region of SEQ ID NO: 3, the LCDR1 region of SEQ ID NO: 4, the LCDR2 region of SEQ ID NO: 5 and the LCDR3 region of SEQ ID NO: 6 for use in treating, inhibiting and/or reducing the severity of antibody-mediated rejection (ABMR) response of an organ transplant.

In another aspect, the present disclosure provides an anti-CD38 antibody or antibody fragment comprising a variable heavy chain region of SEQ ID NO: 7 and a variable light chain region of SEQ ID NO: 8 for use in treating, inhibiting and/or reducing the severity of antibody-mediated rejection (ABMR) response of an organ transplant in a subject in need thereof.

In a particular aspect, the present disclosure provides MOR202 (felzartamab) for use in treating, inhibiting and/or reducing the severity of antibody-mediated rejection (ABMR) response of an organ transplant in a subject in need thereof.

In one embodiment, the present disclosure provides an anti-CD38 antibody or antibody fragment for use in depleting CD38 expressing antibody secreting cells (preferably plasma cells), in subjects with an antibody-mediated rejection (ABMR) response after having received an organ transplantation.

In a preferred embodiment, the disclosure provides an anti-CD38 antibody (e.g. MOR202) for use in reducing circulating anti-HLA antibodies and/or anti-non-HLA antibodies in subjects with an antibody-mediated rejection (ABMR) response after having received an organ transplantation.

In another embodiment, the disclosure provides an anti-CD38 antibody (e.g. MOR202) for use in reducing deposited anti-HLA antibodies and/or anti-non-HLA antibodies in the graft organ in subjects with an antibody-mediated rejection (ABMR) response after having received an organ transplantation.

In a further aspect, the disclosure provides a therapeutic agent comprising an anti-CD38 antibody (e.g. MOR 202) as an active ingredient for use in reducing the symptoms of ABMR in a subject after having received a kidney transplantation, wherein the symptom is selected from: (i) aggravation of kidney function measured by serum creatinine and estimated glomerular filtration rate (eGFR); (ii) presence of donor specific antibodies; and/or (iii) capillaritis, inflammation and complement (C4d) deposition in the kidney.

In another aspect, the disclosure provides a preventive and/or therapeutic agent comprising an anti-CD38 antibody (e.g. MOR202) for use in restoring, ameliorating or normalizing kidney function indicated by glomerular filtration rate (eGFR) based on the CKD-epi equation in subjects with an antibody-mediated rejection (ABMR) response after having received a kidney transplantation.

In a further aspect, the disclosure provides an anti-CD38 antibody (e.g. MOR202) for use in the treatment of ABMR response of an organ transplant in a subject in need thereof, whereby the anti-CD38 antibody (e.g. MOR202) will be dosed in at least 2 doses, at least 5 doses, at least 7 doses or at least 9 doses.

In another aspect, the disclosure provides an anti-CD38 antibody (e.g. MOR202) for use in the treatment of ABMR response of an organ transplant in a subject in need thereof, whereby the anti-CD38 antibody (e.g. MOR202) will be dosed in 2 doses, in 5 doses, in 7 doses or in 9 doses.

In a specific embodiment, dosing will be at 8 mg/kg or more. In a particular embodiment, dosing will be at 16 mg/kg.

In another embodiment, the disclosure provides an anti-CD38 antibody for use in the treatment of ABMR of an organ transplant in a subject in need thereof, wherein said antibody is administered every two weeks in cycle 1 (C1) and every four weeks in cycles 2-6 (administration of felzartamab/placebo at day 0 and 14 (cycle 1), and thereafter in 4-weekly intervals at weeks 4, 8, 12, 16, and 20 (cycles 2-6).

In another embodiment, the disclosure provides an anti-CD38 antibody for use in the treatment of ABMR, wherein said anti-CD38 antibody is administered intravenously.

In another embodiment, the disclosure provides an anti-CD38 antibody for use in the treatment of ABMR, wherein said antibody is administered intravenously over a period of two hours.

In one embodiment, the anti-CD38 antibody (e.g. MOR202) is administered before, concurrently with, and/or after the organ transplantation.

In another embodiment, methods for treating an individual in need of transplantation by administering to the individual an effective amount of felzartamab before, concurrently with, and/or after the transplantation.

In another aspect, the present disclosure provides the use of an anti-CD38 antibody or antibody fragment in the preparation of a medicament for the treatment and/or prophylaxis of an antibody-mediated rejection (ABMR) response of an organ transplant in a subject in need thereof.

In other aspects, the present disclosure provides the use of an anti-CD38 antibody or antibody fragment comprising the HCDR1 region of SEQ ID NO: 1, the HCDR2 region of SEQ ID NO: 2, the HCDR3 region of SEQ ID NO: 3, the LCDR1 region of SEQ ID NO: 4, the LCDR2 region of SEQ ID NO: 5 and the LCDR3 region of SEQ ID NO: 6 in the preparation of a medicament for the treatment and/or prophylaxis of an antibody-mediated rejection (ABMR) response of an organ transplant in a subject in need thereof.

In other aspects, the present disclosure provides the use of an anti-CD38 antibody or antibody fragment comprising a variable heavy chain region of SEQ ID NO: 7 and a variable light chain region of SEQ ID NO: 8 in the preparation of a medicament for the treatment and/or prophylaxis of an antibody-mediated rejection (ABMR) response of an organ transplant in a subject in need thereof.

In a further aspect, the present disclosure provides the use of MOR202 (felzartamab) in the preparation of a medicament for the treatment and/or prophylaxis of an antibody-mediated rejection (ABMR) response of a kidney transplant in a subject in need thereof.

In other aspects, the present disclosure provides the use of MOR202 (felzartamab) or pharmaceutical compositions comprising MOR202 (felzartamab), in combination with another therapeutic agent, in the preparation of a medicament for the treatment and/or prophylaxis of an antibody-mediated rejection (ABMR) response of an organ transplant in a subject in need thereof.

In some embodiments, the use of MOR202 in combination with a steroid for the treatment and/or prophylaxis of ABMR in a subject in need thereof is provided. In other aspects, MOR202 is administered in combination with a proteasome inhibitor, (e.g. bortezomib or carfilzomib) for use in the treatment and/or prophylaxis of ABM R.

In one aspect, the present disclosure provides a method for the treatment and/or prophylaxis of an antibody-mediated rejection (ABMR) response of an organ transplant in a subject in need thereof, comprising administering to said subject an anti-CD38 antibody. In a particular embodiment, the antibody-mediated rejection (ABMR) response is directed against a kidney graft.

In some embodiments, the present disclosure provides methods of prophylaxis and/or treatment of subjects suffering from an antibody-mediated rejection (ABMR) response of an organ transplant, wherein said subject is resistant to treatment by other immunosuppressant therapies, comprising corticosteroids or calcineurin inhibitors or B cell depleting therapies (e.g. with Rituximab or any other anti-CD20 antibody, or anti-BAFF antibody), which methods comprise the administration of an effective amount of an anti-CD38 antibody or antibody fragment.

In one aspect, the disclosure provides methods of using an anti-CD38 antibody or antibody fragment to achieve a prophylactic or therapeutic benefit in patients susceptible or vulnerable to an antibody-mediated rejection (ABMR) response after receiving an organ transplant.

In another aspect, the disclosure provides a method for reducing the incidence of an antibody-mediated rejection (ABMR) response, ameliorating an antibody-mediated rejection (ABMR) response, suppressing an antibody-mediated rejection (ABMR) response, palliating an antibody-mediated rejection (ABMR) response, and/or delaying the onset, development, or progression of an antibody-mediated rejection (ABMR) response, and/or its symptoms in a subject, said method comprising administering an effective amount of an anti-CD38 antibody to the subject. Particularly, the antibody-mediated rejection (ABMR) response is after a kidney transplantation.

In preferred embodiments, the disclosure provides methods for treating patients with elevated levels of DSA associated with the antibody-mediated rejection (ABMR) response.

In other aspects, the present disclosure provides a method for the treatment and/or prevention of a disease caused by the presence of donor-specific antibodies. In yet other aspects, the present disclosure provides a method for the treatment and/or prevention of symptoms associated with the presence of anti-donor HLA antibodies. In further aspects, the present disclosure provides a method for the treatment and/or prevention of symptoms associated with the presence of anti-donor antibodies that are not directed against HLA.

In other embodiments, the disclosure provides methods to reduce inflammation and C4d complement deposition in subjects suffering from an antibody-mediated rejection (ABMR), which methods comprise the administration of an effective amount of an anti-CD38 antibody or antibody fragment or one or more of the pharmaceutical compositions herein described. For example, the methods provided herein comprise administering an anti-CD38 antibody to patients with elevated levels of anti-HLA antibodies. In other aspects, the methods provided herein comprise administering an anti-CD38 antibody to patients with elevated levels of C4d complement deposits in the transplanted organ.

In one embodiment, the reduction (change) of anti-HLA levels in serum of subjects suffering from antibody-mediated rejection (ABMR) is at least 5%, at least 10%, at least 15%, at least 20%, at least 25%, at least 30%, at least 35%, at least 40%, at least 45%, or at least 50% compared to baseline after administering an anti-CD38 antibody or antibody fragment, or one or more of the pharmaceutical compositions herein described.

In another aspect, the disclosure provides methods for preventing the decline of renal function in an individual with antibody-mediated rejection (ABMR), which methods comprise the administration of an effective amount of an anti-CD38 antibody, or antibody fragment, or one or more of the pharmaceutical compositions herein described.

In further embodiments, the present disclosure refers to a method for the treatment of antibody-mediated rejection (ABMR), in a subject, comprising administering to the subject a pharmaceutical composition comprising an anti-CD38 antibody or antibody fragment that binds to a CD38 expressing cell and leads to the depletion of such CD38 expressing cell.

In a preferred embodiment, the present disclosure refers to a method for the treatment of ABMR in a subject, comprising administering to the subject a pharmaceutical composition comprising an anti-CD38 antibody or antibody fragment that binds to a CD38 expressing antibody-secreting cell and leads to the depletion of said antibody-secreting cell, while sparing regulatory T cell and/or B cell populations.

In another embodiment, the present disclosure refers to a method for the treatment of ABMR in a subject, comprising administering to the subject a pharmaceutical composition comprising an anti-CD38 antibody or antibody fragment that binds to a CD38 expressing antibody-secreting cell and leads to the depletion of said antibody-secreting cell, but does not lead to a significant depletion of regulatory T cells.

In a particular preferred embodiment, the present disclosure refers to a method for the treatment of antibody-mediated rejection (ABMR) in a subject, comprising administering to the subject a pharmaceutical composition comprising an anti-CD38 antibody or antibody fragment that binds to a CD38 expressing antibody-secreting cell and leads to the depletion of such CD38 expressing antibody-secreting cell, wherein the antibody shows a significant higher specific cell killing on antibody-secreting cells than on NK cells.

In one embodiment, the present disclosure refers to a method for the treatment of antibody-mediated rejection (ABMR) in a subject, comprising administering to the subject a pharmaceutical composition comprising an anti-CD38 antibody or antibody fragment that binds to a CD38 expressing antibody-secreting cell and leads to the depletion of such CD38 expressing antibody-secreting cell, wherein the specific cell killing of the antibody-secreting cell is at least 10%, at least 15%, at least 20%, at least 25%, at least 30%, at least 35%, at least 40% and wherein the specific cell killing of antibody-non-secreting NK cells is less than 30%, less than 25%, less than 20%, or less than 15% as determined in a standard ADCC assay.

In one embodiment, the present disclosure refers to a method for the treatment of antibody-mediated rejection (ABMR) in a subject, comprising administering to the subject a pharmaceutical composition comprising an anti-CD38 antibody or antibody fragment, wherein the subject has undergone standard-of-care treatment comprising one or more of immunoglobulin administration (IVIG), rituximab administration and plasma exchange (PLEX), and the subject's response to the standard- of-care treatment is ineffective.

In another embodiment the subject to be treated is further resistant or has acquired resistance to immunosuppressive treatment with one or more of eculizumab, thymoglobulin, bortezomib, carfilzomib, basiliximab, mycophenolate mofetil, tacrolimus and corticosteroids.

In another embodiment, the present disclosure refers to a method for the treatment of antibody-mediated rejection (ABMR) in a subject, comprising administering to the subject a pharmaceutical composition comprising an anti-CD38 antibody or antibody fragment, wherein the subject has not undergone any prior standard-of-care treatment.

In another embodiment, the present disclosure refers to a method for the treatment of ABMR in a subject, comprising administering to the subject a pharmaceutical composition comprising an anti-CD38 antibody or antibody fragment, wherein administration of the anti-CD38 antibody does not result in a significant change the absolute number of regulatory CD4+, CD25+, CD127-T cells.

In another embodiment, the present disclosure refers to a method for the treatment of ABMR in a subject, comprising administering to the subject a pharmaceutical composition comprising an anti-CD38 antibody or antibody fragment, wherein the CD8+T cell/Treg ratio does not increase significantly after antibody administration.

In another embodiment, the present disclosure refers to a method for the treatment of ABMR in a subject, comprising administering to the subject a pharmaceutical composition comprising an anti-CD38 antibody or antibody fragment, wherein administration of said anti-CD38 antibody or antibody fragment leads to a reduction of class I and/or class II anti-HLA antibody levels. Anti-HLA class I antibodies comprise anti-HLA-A, -B, and —C. Anti-HLA class II antibodies comprise anti-HLA-DR, -DQ (e.g. anti-DQ5), and -DP.

In a preferred embodiment, the method for the treatment of ABMR is in a human subject and comprises administration of a pharmaceutical composition comprising MOR202 (felzartamab), wherein said administration leads to a reduction of class II anti-HLA antibody levels, preferably anti-DQ5 antibody levels. In a preferred aspect, 9 doses of MOR202 are administered.

### WORKING EXAMPLES

### Example 1: Felzartamab in Late Antibody-Mediated Renal Allograft Rejection

### 1.1 Study Design

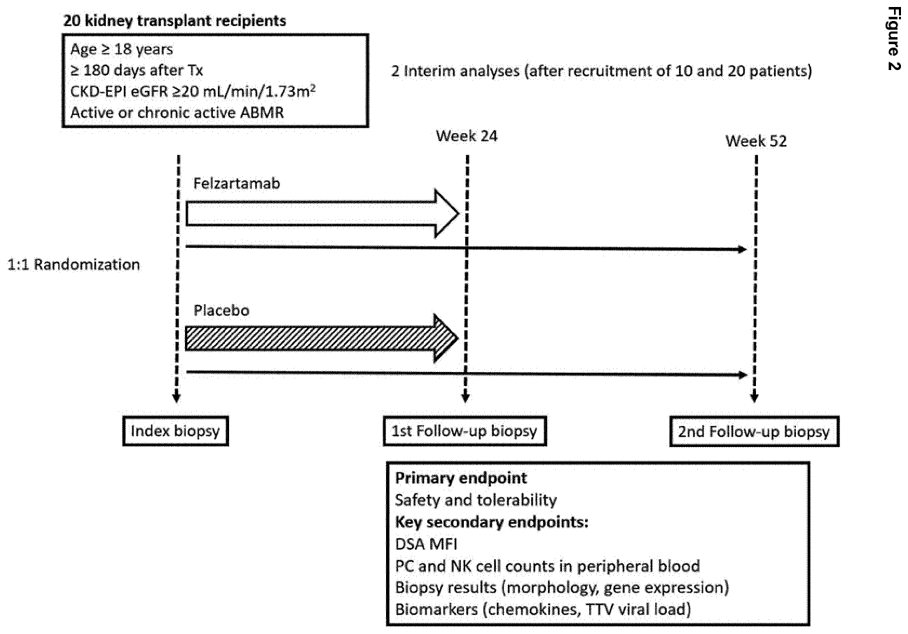

This study is an investigator-driven pilot trial designed to assess safety, tolerability, pharmacokinetics, pharmacodynamics and efficacy of the fully human anti-CD38 monoclonal antibody felzartamab in kidney transplant recipients with late active or chronic-active ABMR. The trial is designed as a randomized, controlled, double-blind phase 2 pilot trial. The primary endpoint will be safety and tolerability. A simplified flow chart of the trial is shown in FIG. 2.

### 1.2 Study Population

About 20 kidney transplant recipients with circulating anti-HLA DSA and biopsy features of late (≥80 days post-transplant) active ABMR (according to the Banff 2019 scheme) in an indication biopsy (index biopsy; performed within the clinical routine for a positive post-transplant DSA result and slow deterioration of allograft function and/or proteinuria) will be included. Other key inclusion criteria are an age >18 years a functioning graft at ≥180 days post-transplantation and an estimated GFR (eGFR according to the CKD-EPI equation) ≥20 ml/min/1.73 m². Inclusion and exclusion criteria are detailed in Table 1.

TABLE 1
Inclusion and exclusion criteria.

INCLUSION CRITERIA:
Voluntary written informed consent
Age >18 years (maximum: 70 years)
Functioning living or deceased donor allograft after ≥180 days post-transplantation
eGFR ≥20 ml/min/1.73 m² (CKD-EPI formula)
HLA class I and/or II antigen-specific antibodies (preformed and/or de novo DSA).
Active or chronic/active ABMR (±C4d in PTC) according to the Banff 2019 classification
Molecular ABMR score (MMDx) ≥0.2
EXCLUSION CRITERIA:
Patients actively participating in another clinical trial
Age ≤18 years
Female subject is pregnant or lactating or not on adequate contraceptive therapy
ABO-incompatible transplant
Index biopsy results:
T-cell-mediated rejection classified Banff grade ≥ I
De novo or recurrent severe thrombotic microangiopathy
Polyoma virus nephropathy
De novo or recurrent glomerulonephritis
Acute rejection treatment ≤3 month before screening
Previous treatment with other CD38 monoclonal antibodies (e.g. daratumumab)
Previous treatment with other immunomodulatory monoclonal/polyclonal antibodies (e.g.
CD20 Ab rituximab, IL-6/IL-6R Ab) ≤3 months before study treatment
Total bilirubin >2 × the upper limit of normal [ULN], alanine transaminase and aspartate
aminotransferase >2 · 5 × ULN
Haemoglobin <8 g/dL
Thrombocytopenia: Platelets <100 G/L
Leukopenia: Leukocytes <3 G/L
Neutropenia: Neutrophils <1.5 G/L
Hypogammaglobulinemia: Serum IgG <400 mg/dL
Active viral, bacterial or fungal infection precluding intensified immunosuppression
Active malignant disease precluding intensified immunosuppressive therapy
Latent or active tuberculosis (positive QuantiFERON-TB-Gold test)
Administration of a live vaccine within 6 weeks of screening
History of alcohol or illicit substance abuse
Serious medical or psychiatric illness likely to interfere with participation in the study

### 1.3 Dosing

Subjects will be randomized 1:1 using a web-based randomization platform (www.meduniwien.ac.at/randomizer) to receive either felzartamab (16 mg/kg, i.v. administration) or placebo. Based on the results of a PK modelling for an ongoing phase Ib/Ila trial in autoimmune disease (membranous nephropathy, ClinicalTrials.gov, NCT04145440), patients will be dosed with felzartamab for a period of 6 months, administered as an intravenous infusion. As (in contrast to patients with membranous nephropathy) transplant patients are on multi-compound immunosuppressive baseline therapy, and therefore at increased risk of infections, an extension of dosing intervals for the first cycle (every 2 weeks instead of every week) is planned. Instead of 9 doses, 7 doses of felzartamab will be administered i.v. at 16 mg/kg over 6 treatment cycles at 28-days each. Dosing occurs every two weeks in cycle 1 (C1) and every four weeks in cycles 2-6 (administration of felzartamab/placebo at day 0 and 14 (cycle 1), and thereafter in 4-weekly intervals at weeks 4, 8, 12, 16, and 20 (cycles 2-6).

In a preferred setting, subjects will be randomized to receive either felzartamab (16 mg/kg, intravenous administration) or placebo (0.9% saline) (1:1 randomization) for a period of 6 months (administration of felzartamab/placebo at day 0, 7, 14, 21 (cycle 1), and thereafter in 4-weekly intervals at weeks 4, 8, 12, 16, and 20 (cycles 2-6). After six (week 24) and twelve months (week 52), study participants will be subjected to follow-up allograft biopsies. Primary goals of the trial are to assess the safety, pharmacokinetics and pharmacodynamics (peripheral blood PC and NK cell depletion) of a 6-month course of treatment over a period of 12 months.

Thus, 9 doses of felzartamab or placebo as an intravenous infusion are applied over 6 treatment cycles at 28 days each. Dosing occurs every week in cycle 1 and every four weeks in cycles 2-6.

Felzartamab will be supplied at 65 mg/mL in 10 mM Histidine, 260 mM Sucrose, 0.1% Tween 20, pH 6.0 after reconstitution with 4.8 mL water for injection (One vial contains 325 mg MOR202). Felzartamab will be administered after dilution with 250 mL 0.9% sodium chloride solution (final concentration should be between 1 and 20 mg/mL). Placebo (0.9% sodium chloride) will be administered with 250 mL normal saline for infusion. Prepared infusions may be stored for up to 24 hours at 2° C. to 8° C., and up to 4 hours of the 24 hours at room temperature, 15° C. to 25° C. Prior to administration, felzartamab/placebo infusion must reach room temperature by storing unrefrigerated for 30 to 60 minutes before use.

The first two infusions of felzartamab will be slow (approximately 90 min), and, if no infusion reactions occur, infusion times may be shortened to 1 hour or shorter (minimum 30 min) in subsequent infusions.

After six (week 24) and twelve months (week 52), study participants will be subjected to follow-up allograft biopsies. Randomization will be according to ABMR categories (active ABMR versus chronic/active ABMR) to ensure a balance of patients with these two histological types between the two arms. The study is designed as a double-blinded trial, in order to minimize bias.

### Premedication

To prevent infusion-related reactions, patients allocated to the felzartamab arm will receive i.v. premedication prior to the first two felzartamab infusions (day 0 and day 14). Patients in the placebo arm will receive placebo (0.9% NaCI solution). Premedication will be administered 30 min before the infusion of felzartamab, and will consist of Diphenhydramine (30 mg), Paracetamol (1000 mg), and Prednisolon (100 mg), respectively (each in 100 mL Volume). In the placebo arm, patients will receive 3x100 mL NaCI 0.9%.

The following medications are prohibited during the study:

Rituximab, eculizumab, proteasome inhibitors, IVIG, plasma exchange or immunoadsorption, other investigational drugs/treatments including commercially available CD38 or anti-IL-6/sIL-6R monoclonal antibody drugs such as daratumumab (Darcalex®) or tocilizumab (RoActemra®/Actemra®).

The following concomitant medications are permitted during the study:

Calcineurin inhibitors (CNI, tacrolimus or cyclosporine A), mammalian target of rapamycin (mTOR) inhibitor (everolimus or rapamycin), Mycophenolate mofetil (MMF)/mycophenolate sodium; long-term treatment with low dose corticosteroids (prednisolone 5mg/day).

Baseline immunosuppression: Upon diagnosis of late ABMR, all recipients on therapy with a calcineurin inhibitor [tacrolimus or cyclosporine A (CyA)] or a mTOR inhibitor (everolimus or rapamycin), without azathioprine or mycophenolic acid (MPA), will receive mycophenolate mofetil (or, alternatively, enteric-coated mycophenolic acid (EC-MPA), initially at a dose of 2×500 mg (or 2×360 mg, respectively) per day; stepwise increase to 2×1000 mg (or 2×720 mg) per day if tolerated) to avoid under-immunosuppression. Tacrolimus will be adjusted to achieve target trough levels between 5 and 10 ng/mL, CyA to 80-120 ng/mL. Recipients weaned off steroids will receive low dose prednisolone (5 mg/day).

### 1.4 Efficacy assessment

Primary goals of the trial are to assess safety, pharmacokinetics and pharmacodynamics (peripheral blood PC and NK cell depletion) of a 6-month course of treatment over a period of 12 months. Further, data on efficacy (progression/activity of rejection, blood biomarkers) and potential associations of treatment with parameters reflecting clinical progression of allograft dysfunction (e.g.: course of renal function as described in Irish, Wet al. 2020, Transplantation, or iBOX score Loupy, A et al, BMJ, 366: 14923, 2019) will be provided.

TABLE 2
Study endpoints

PRIMARY OUTCOME
Safety and tolerability of felzartamab in renal allograft recipients with ABMR on baseline
immunosuppression
SECONDARY OUTCOMES
DSA/immunoglobulin levels (week 0, 12, 24, and 52)
Mean fluorescence intensity (MFI) of immunodominant DSA
Changes in levels of the immunodominant DSA calculated form dilution experiments
Number of detected DSA
Total Ig (IgG, IgA, IgM) and IgG subclasses (IgG1, IgG2, IgG3, IgG4)
Course of vaccination titers
Effect on leukocyte subsets in peripheral blood (week 0, 12, 24, and 52) or (week 0, 1, 4,
8, 12, 24, and 52)
Circulating PC, NK cells, T and B cell subpopulations, expression of CD38 using non-
crossreactive CD38 Ab clone HIT2
Results of follow-up protocol biopsies (week 24 and 52)
Morphological results:
ABMR category (active vs. chronic active ABMR; C4d+ vs. C4d− ABMR)
Extent of glomerular/pertibulular capillary microcirculation inflammation (g + ptc score)
Transplant glomerulopathy (cg) and interstitial fibrosis/tubular atrophy scores
Intragraft complement activation
Patterns of intragraft cellular infiltrates (NK cells, PC, T cells, B cells)
Gene expression analysis (Molecular Microscope ® Diagnostic System, MMDx)
Molecular classifiers/scores related to ABMR and T cell-mediated rejection
Molecular classifiers/scores related to rejection in general
Molecular classifiers/scores related to acute and chronic renal injury
Archetypal analysis of rejection-related categories
Pathogenesis-based transcripts (PBT) scores (cytotoxic T cell infiltration, γ-interferon
effects, NK cell burden, epithelial cell damage)
Effect on rejection or immunologic biomarkers (week 0, 12, 24, and 52)
CXCL9 and CXCL10 levels in blood and urine (Luminex-based detection)
BAFF levels in blood (ELISA/Luminex)
Effect on measures of overall immunosuppression (week 0, 12, 24, and 52)
Torque Teno virus (TTV) levels in plasma (quantitative PCR)
Effect on clinical outcome parameters and surrogate endpoints:
eGFR slope over a period of 52 weeks (4-weekly measurements)
iBox clinical prediction score at 12, 24 and 52 weeks
Protein excretion (protein/creatinine ratio) over 52 weeks (4-weekly measurements)
12-month (death-censored and overall) graft and patient survival

Major endpoints (see Table 2) include safety and tolerability, the course of DSA (and in parallel total Ig and IgG subclass levels), the dynamics of peripheral blood counts of PC, NK cells, and T and B cell subpopulations (assessed by FACS), as well as biomarkers of rejection (CXCL9 and CXCL10 in blood and urine) and overall immunosuppression (Torque Teno viral load). Moreover, 6- and 12-month renal allograft biopsies will be assessed for morphological (Banff criteria of rejection and chronic injury; immunohistochemistry for detection of complement activation/deposition and characterization of cellular infiltrates including NK cells) and molecular rejection criteria (molecular ABMR score; microarray analysis using the Molecular Microscope® Diagnostic System), including pathogenesis-based transcripts (PBT) scores (cytotoxic T cell infiltration, γ-interferon effects, natural killer cell burden, epithelial cell damage) in 6- and 12 month biopsies. Clinical endpoints will be proteinuria as well as the slope of eGFR and the iBox clinical prediction score, both validated surrogate endpoints that accurately predict long-term allograft survival.

### Example 2: Experimental Methods

### 2.1 HLA antibody detection

For assessment of HLA antibody levels, serum samples will be evaluated after completion of the study according to published protocols (Doberer, K et al.; J Am Soc Nephroi). In brief, LABscreen single-antigen flow-bead assays (One Lambda) will be applied for antibody detection. Serum samples will be incubated with 10 mM EDTA to prevent complement interference. Data acquisition will be performed via a LABScanTM 200 flow analyzer (Luminex Corporation). For longitudinal analysis of DSA/HLA antibody levels, bead assays will be performed retrospectively to avoid influences of day-by-day variations in test results. Donor-specificity will be defined according to serological and/or low- or high-resolution donor/recipient HLA typing (HLA-A, -B, -Cw, -DR, -DQ, -DP). Test results will be documented as mean fluorescence intensity (MFI) of the immunodominant DSA. An MFI threshold >1,000 will be considered as positive. Impact of felzartamab treatment on DSA levels, will be estimated by the percent change in MFI. To quantify changes in DSA levels more accurately, additional dilution experiments will be performed following the methods as described in Doberer K et al. 2020, Transplantation. In brief, nonlinear standard curves based on raw DSA MFI levels (immunodominant DSA) will be obtained by serial dilution of individual patient sera collected prior to start of treatment (all samples were incubated with EDTA) and at week 24. According to computed standard curves, the fold change of antibody levels will then be calculated from DSA MFI levels detected in the same experiment for undiluted week-12, -24 and week-52 samples.

### 2.2 Immunoglobulin Levels

Total IgG, IgM and IgG subclasses will be assessed in serum applying immunonephelometry on a BN™ II analyzer (Siemens Healthineers).

### 2.3 Transplant biopsies

Follow-up biopsies will be performed at weeks 24 and 52 (end-of-study visit), after exclusion of a coagulation disorder or platelet counts below 80%. The biopsy will be performed under local anaesthesia (lidocain) using ultrasound-guided percutaneous techniques. Histomorphology will be evaluated on paraffin-embedded sections applying standard methodology. The embedded tissue blocks undergo serial sectioning (5-mm thick) and staining for hematoxylin and eosin and periodic acid—Schiff for routine evaluation and grading for rejection. For immunohistochemical C4d staining, a polyclonal anti-C4d antibody (BI-RC4D, Biomedica) will be used and following the Banff scheme (Loupy, A et al. 2020, American Journal of Transplantation: ajt.15898) minimal immunohistochemical staining (C4d Banff score ≥1) along peritubular capillaries will be considered positive. Biopsies will also be evaluated by electron microscopy for detection of multilayering of peritubular capillary basement membranes (MLPTC). In addition, all biopsies will be analyzed using microarrays as also proposed by the Banff scheme, using the internationally validated Molecular Microscope® Diagnostic System MMDx platform. Thoroughly validated molecular scores based on machine-learning derived lesion-based classifiers related to rejection [ABMR, T cell-mediated rejection (TCMR), all Rejection], inflammation (global disturbance score) or chronic injury (atrophy/fibrosis score) will be generated using a reference set of 1529 biopsies.

For classification of ABMR according to the Banff 2019 scheme, all biopsy results will be analyzed in the context of the molecular results. ABMR will be defined based on both morphological (histomorphology, immunohistochemistry, electron microscopy) and thoroughly validated molecular criteria: (i) evidence of acute or chronic tissue injury, (ii) evidence of current/recent antibody interaction with the vascular endothelium, and (iii) serological evidence of DSA.

### 2.4 Kidney Function

eGFR will be assessed using the Chronic Kidney Disease Epidemiology Collaboration (CKD-EPI) equation (mL/min/1.73m²). Protein excretion will be documented as protein/creatinine ratio in spot urine (mg/g).

### 5 2.5. Immunologic Biomarkers of Rejection

For chemokine detection a Luminex-based protocol as described by MOhlbacher, J, et al. (2020, Front Med, 7: 114) will be used. For quantification of chemokine (C-X-C motif) ligand (CXCL)9 and CXCL10, serum samples will be adjusted to 10 mM EDTA to prevent complement interference. Undiluted samples will be measured in duplicates using multiplexed Human ProcartaPlex Simplex Immunoassays (Thermo Fisher Scientific) according to the manufacturer's instructions. Immunoassays will be performed on a Luminex 200 instrument (Luminex Corp.). Urinary results will be normalized to creatinine excretion and presented as pg (chemokine)/mg (creatinine). Levels of dd-cf DNA in recipient plasma samples reflecting the extent of ongoing allograft injury will be detected using standard technology, based on the detection of a defined set of single nucleotide polymorphisms detected by next-generation sequencing on an Illumina MiSeq sequencer (Illumina Inc).

### 2.6 Immune Cell Monitoring and Leukocyte Subpopulations

The underlying mechanisms of chronic antibody mediated rejection, especially the role of peripheral T- and B-cell subsets are not fully clarified. Thus, the prospective monitoring of immune phenotype under therapy with felzartamab is a promising approach to elucidate the impact on immune-regulatory pathways when CD38 is targeted. Moreover, assessment of plasma cell and NK cell counts allows for the monitoring of the pharamacodynamic effects of the anti-CD38 antibody. For monitoring of leukocyte (sub) populations, reproducible immune monitoring panels for phenotyping will be used (e.g.: DuraClone® for flow cytometry). In the DuraClone kits pre-defined assay tubes contain a layer with the dried-down antibody panel ready to use. Up to 10 different monoclonal antibodies per tube allows the identification of leukocyte (e.g. T cell, B cell, NK cell subsets) subpopulations present in whole blood samples.

For monitoring immune cells, cells from blood, lymph nodes, bone marrow, spleen, and graft are stained with the LIVE/DEAD Fixable Aqua Dead Cell Stain Kit (Life Technologies). Then cells will 30 be stained with one or more of the following mAbs against human: CD3, CD4, CD8, CD14, CD20,

CD25, CD27, CD28, CD38, CD56, CD95, CD127, CD159a, CD278 (ICOS), CD279 (PD-1), IgM, IgG, CXCR5, and—after fixation—Ki67 and FoxP3. Samples are collected by a flow cytometer and analyzed using a standard software (e.g. FlowJo v9.6.) for percentages of CD38+B cells and plasma cells, CD8+ T cells and/or CD4+, CD25+, CD127-T cells.

### 35 2.7 Gene expression analysis

For gene expression analysis, 5 mL of blood will be collected in PAXgene Blood RNA tubes and stored at −80° C. until retrospective analysis. These tubes are designed for stabilization of RNA in blood during long-term storage at ultra-low temperature. Gene expression pattern analyses (microarray analysis) will be performed from peripheral blood to evaluate the impact of felzartamab on antibody-producing cells, analyzing genes annotated as part of the B-cell receptor signalling pathway.

### 5 2.8 Torque Teno virus (TTV) quantification

For TTV analysis, DNA is extracted from plasma samples using the NucliSENS easyMAG platform (bioMerieux) and eluted in 50 μL of elution buffer. TTV DNA will be quantitated by TaqMan real time PCR, as described e.g. by Schiemann, M et al. (2017, Transplantation, 101: 360-367). Quantitative PCRs will be performed in a volume of 25 μL using 2×TaqMan Universal PCR Master Mix, containing 5 μL of extracted DNA, 400 nM of each primer, and 80 nM of the probe. Thermal cycling will be started for 3 minutes at 50° C., followed by 10 minutes at 95° C., and then by 45 cycles at 95° C. for 15 seconds, at 55° C. for 30 seconds, and at 72° C. for 30 seconds, using the CFX96 Real-time System (Bio-Rad). Results will be recorded as copies/mL.

### 2.9 Course of Vaccination Titers

Serum IgG titers specific for mumps, measles and rubella (MMR) will be analyzed by standard ELISA technique.

### 2.10 Collection of biological material (outside routine monitoring)

Plasma (10 mL; chemokines, TTV load), serum (10 mL; HLA antibody studies), whole blood (10 mL; flow cytometry, RNA for gene expression analysis) and urine (10 mL) will be collected before study initiation (day 0), after 6 and after 12 months (3x30 mL peripheral blood). Finally, for measurement of felzartamab concentrations and ADA, serum will be obtained at every study visit (5 mL peripheral blood per visit; total of 18 visits).

### Example 3: Safety and efficacy of MOR202 to prevent and treat ABMR in nonhuman primates undergoing kidney transplantation

### 25 3.1 Experimental NHP Model

This study is to investigate the safety and efficacy of MOR202 on desensitization (e.g. lowering preformed antibody), preventing ABMR and acute post-transplant ABMR in a highly sensitized nonhuman primate kidney transplant model (see Kwun J. et al. J Am Soc Nephrol. 2019 Jul.;30(7):1206-1219). Further, the longer-term effect of MOR202 on preventing rebound donor-specific antibody (DSA) and late/chronic ABMR is evaluated.

### 3.1.1 CD38 expression

Expression level of CD38 on plasma cells from BM, spleen, lymph nodes, and blood of recipient animals and cross-reactivity with MOR202 will be analyzed. CD38 expression levels on red blood cells will be checked to estimate risk of anemia.

### 3.1.2 Desensitization with MOR202

For allosensitization, male rhesus macaques (Macaca mulatta) will be sensitized to MHC-mismatched donors by two successive skin grafts placed at 8-week intervals, as described in Burghuber CK et al. Am J Transplant 19: 724-736. Approximately, 8-12 weeks after the second skin graft, monkeys are treated with MOR202 at 16 mg/kg for 4 weeks. Then alloantibody levels are measured. The level of desensitization will be compared to the results of the desensitization strategy using proteasome inhibitor (bortezomib/carfilzomib) alone or in combination with costimulation blockade (Kwun J. et al. Blood Adv. 2017 Nov 14; 1(24): 2115-2119). CMV titers will be measured before and after completing drug treatment. For monitoring immune cells, cells from blood, lymph nodes, bone marrow, spleen, and graft will be assessed by flow cytometry.

### 3.1.3 Efficacy of MOR202 to prevent and treat ABMR after desensitization treatment

Animals will undergo renal transplantation from their same skin graft donor, and in addition to anti-rejection immunosuppression with rATG, tacrolimus, steroid, they will also receive MOR202 weekly for 4 weeks. Renal transplants will be performed basically as described by Burghuber CK, et al (Am J Transplant. 2016;16(6):1726-1738). For depletion of plasma cell populations, sensitized rhesus monkeys will be treated weekly with MOR202. Control animals received no treatment prior to kidney transplantation. Because CD38 is expressed in hematopoietic and nonhematopoietic cells, including activated B cell and T cell populations, circulating B and T cell populations will be evaluated by FACS. These include circulating B cells, IgG+B cells, and memory B cells (IgG+CD27+CD20+), as well as naive (CD28+CD95-), central memory

(CD28+CD95+), and effector memory (CD28-CD95int) subsets of CD4 and CD8 T cells.

Kidneys biopsies will be collected at 1 month, 3 months, 6 months and at sacrifice and analyzed by (Immuno)Histology and scored according to the Banff criteria. Donor specific antibodies (DSA) after transplantation will be measured weekly thereafter. Animals with rebound DSA showing elevated serum creatinine will also be treated with MOR202 for one month. Cellular and humoral immune responses will be analyzed including follicular help T cells, plasma cells (BM, LN, and blood), and plasmablasts (blood and LNs). Additional kidney graft biopsies will be collected as needed. H&E, PAS and C4d staining will be performed to monitor for subclinical rejection and C4d deposition.

### 30 3.1.4 DSA Monitoring

DSA levels will be continuously measured weekly via flow crossmatch using donor lymphocytes and recipient serum as described by Burghuber CK et al. (Am J Transplant 19: 724-736). Briefly, donor PBMC or splenocytes will be incubated with recipient serum, washed, and stained with FITC-labeled anti-monkey IgG, anti-CD20 mAb and anti-CD3 mAb. Mean fluorescence intensity 35 (MFI) of anti-monkey IgG on T cells or B cells will be measured and expressed as MFI change from presensitized time point. NHP serum alloantibody may also be measured using a human solid phase Luminex assay that uses single HLA antigen beads (LABScreen Single Antigen; One Lambda) to detect crossreactive antibodies.

1. An anti-CD38 antibody or antibody fragment for use in the treatment and/or prevention of antibody-mediated rejection of an organ transplant in a human subject.

2. An anti-CD38 antibody or antibody fragment for use according to claim 1, wherein the organ transplant is a kidney, heart, liver, lung, pancreas, stomach, skin or intestine transplant.

3. An anti-CD38 antibody or antibody fragment for use according to claim 1 or claim 2, wherein the antibody comprises a HCDR1 region of amino acid sequence SEQ ID NO.: 1, a HCDR2 region of amino acid sequence SEQ ID NO.: 2, a HCDR3 region of amino acid sequence SEQ ID NO.: 3, and a LCDR1 region of amino acid sequence SEQ ID NO.: 4, a LCDR2 region of amino acid sequence SEQ ID NO.: 5 and a LCDR3 region of amino acid sequence SEQ ID NO.: 6.

4. An anti-CD38 antibody or antibody fragment for use according to claim 3, wherein anti-CD38 said antibody or antibody fragment comprises a variable heavy chain (VH) region of SEQ ID NO.: 7 and a variable light chain (VL) region of SEQ ID NO.: 8.

5. An anti-CD38 antibody or antibody fragment for use according to any of the preceding claims, wherein said antibody or antibody fragment specific for CD38 is an IgG1.

6. An anti-CD38 antibody or antibody fragment for use according to any of the preceding claims, wherein said antibody or antibody fragment specific for CD38 is a human antibody.

7. An anti-CD38 antibody or antibody fragment for use according to any of the preceding claims, wherein said antibody or antibody fragment specific for CD38 is felzartamab.

8. An anti-CD38 antibody or antibody fragment for use according to any of the preceding claims, wherein the antibody depletes plasma cells by ADCC and/or ADCP.

9. An anti-CD38 antibody or antibody fragment for use according to any of the preceding claims, wherein administration of said anti-CD38 antibody or antibody fragment leads to a reduction of CD38+antibody secreting cells.

10. An anti-CD38 antibody or antibody fragment for use according to any of the preceding claims, wherein administration of said anti-CD38 antibody or antibody fragment leads to a reduction of anti-HLA antibody levels.

11. An anti-CD38 antibody or antibody fragment for use according to claim 10, wherein administration of said anti-CD38 antibody or antibody fragment leads to a reduction of class I and/or class II anti-HLA antibody levels.

12. An anti-CD38 antibody or antibody fragment for use according to claim 11, wherein administration of said anti-CD38 antibody or antibody fragment leads to a reduction of anti-DQ5 antibody levels.

13. The anti-CD38 antibody or antibody fragment for use according to any of the preceding claims, wherein the antibody or antibody fragment is administered i.v. at 16 mg/kg.

14. The anti-CD38 antibody or antibody fragment for use according to claim 13, wherein the antibody or antibody fragment is dosed in at least 2 doses, at least 5 doses, at least 7 doses or at least 9 doses. 20 15. The anti-CD38 antibody or antibody fragment for use according to any of the preceding claims, wherein the subject to be treated is characterized by an eGFR ≥20 ml/min/1.73 m² according the CKD-EPI formula.
